(19) United States
(12) Patent Application Publication
(10) Pub. No.: US 2026/0315760 A1
(43) Pub. Date: Oct. 15, 2026

(54) FISTULIZATION DEVICES, METHODS, AND KITS

(71) Applicant: The University of North Carolina at Chapel Hill, Chapel Hill, NC (US)

(72) Inventors: Alan J. ROSENBAUM, Cary, NC (US); Nicole L. WILEY, Pittsboro, NC (US)

(21) Appl. No.: 19/640,927

(22) Filed: Apr. 7, 2026

Related U.S. Application Data
(60) Provisional application No. 63/786,120, filed on Apr. 9, 2025.

(51) Int. Cl. A61M 27/00

(52) U.S. Cl. CPC A61M 27/00; A61M 2205/0216; A61M 2210/1475

(57) ABSTRACT

A fistulization device includes a central member having opposite proximal and distal ends, at least one first elastomeric arm at the central member distal end, and at least one second elastomeric arm at the central member proximal end. When inserted into a patient through an incision such that the at least one first elastomeric arm extends past a distal opening of the incision, the at least one first elastomeric arm is configured to return to a relaxed state and prevent inadvertent proximal displacement of the device within the incision, and the at least one second elastomeric arm is configured return to a relaxed state and prevent inadvertent distal displacement of the device within the incision. The central member is configured to allow fluid migration past the central member and through a proximal opening of the incision.

### RELATED APPLICATION

This application claims the benefit of and priority to U.S. Provisional Patent Application No. 63/786,120 filed Apr. 9, 2025, the disclosure of which is incorporated herein by reference as if set forth in its entirety.

### FIELD OF THE INVENTION

The present invention relates generally to medical devices and, more particularly, to devices for treating cysts and abscesses.

### BACKGROUND OF THE INVENTION

Bartholin's glands are two small glands located in the vulva whose main function is to secrete mucus to lubricate the vagina and vulva. Under normal conditions the gland is not palpable, but, when the duct becomes blocked, the gland will develop a cyst and further abscess in the range of 2 centimeters (cm) to 4 cm, even 6 cm, causing pain and interrupting day to day life.

The treatment options for such a cyst, ranging from least aggressive/invasive to most aggressive, are incision and drainage (50-60% success rate), fistulization using a Word catheter (80-90% success rate), marsupialization (90-95% success rate), and excision (100% success rate), with the most aggressive options carrying the greatest risk of surgical complications, pain and bleeding. Although utilization of a Word catheter appears to have a high success rate, healthcare providers indicate otherwise, claiming that the device may fall out of a patient too soon, sometimes before they even leave the office after having it placed, resulting in a failed fistulization attempt. In addition, a Word catheter may be difficult to insert after incision and patients may not find it comfortable.

As such, there is a need to find a suitable solution for cyst treatment that healthcare providers can easily implement, and that patients will tolerate for the duration of their treatment.

### SUMMARY OF THE INVENTION

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

Embodiments of the present invention are directed to a fistulization device that can be used in an outpatient or emergent setting for treating Bartholin's cysts and abscesses. The device's unique shape allows for drainage of cyst/abscess contents out of the infected gland, while maintaining its position for the allotted time to allow a fistula to form. The device's small overall shape and soft material are both comfortable and low profile for a patient. Furthermore, removal of the device can be done outpatient without the use of additional tools or pain medication.

Embodiments of the present invention provide a promising improvement to Bartholin's cyst treatment and may decrease procedure time, improve fistulization success rates, and reduce global Bartholin's cyst-related treatment costs. The low-profile device structure of embodiments of the present invention is anticipated to reduce patient discomfort, as well.

According to some embodiments of the present invention, a fistulization device for treating cysts and abscesses includes a flexible member having opposite proximal and distal ends; at least one first elastomeric arm at the flexible member distal end, wherein the at least one first elastomeric arm has a curved configuration when in a relaxed state; and at least one second elastomeric arm at the flexible member proximal end, wherein the at least one second elastomeric arm has a curved configuration when in a relaxed state. Typically, a free end of each of the at least one first elastomeric arm and the at least one second elastomeric arm is blunt (i.e., has an atraumatic shape) so as not to cause damage to patient tissue. Moreover, the outer surface of the fistulization device is typically smooth with no sharp edges.

In some embodiments, the at least one first elastomeric arm includes a pair of first elastomeric arms, and the at least one second elastomeric arm includes a pair of second elastomeric arms. The pair of first elastomeric arms may be arranged in adjacent opposing relationship, and the pair of second elastomeric arms may be arranged in adjacent opposing relationship. For example, the pair of first elastomeric arms curve away from each other when in their respective relaxed states, and the pair of second elastomeric arms curve away from each other when in their respective relaxed states.

In some embodiments, the free end of the at least one first elastomeric arm and the free end of the at least one second elastomeric arm are curved toward each other when in their respective relaxed states.

In some embodiments, the at least one first elastomeric arm, the at least one second elastomeric arm, and the flexible member are substantially within a same plane.

In some embodiments, the flexible member includes at least one longitudinally extending groove in an outer surface thereof. For example, the flexible member may have a plurality of longitudinally extending grooves circumferentially spaced apart around the flexible member.

The fistulization device is flexible (i.e., deformable) so as to be insertable into a lumen of a delivery tool and such that the at least one first elastomeric arm extends longitudinally away from the distal end of the flexible member, and such that the at least one second elastomeric arm extends longitudinally away from the proximal end of the flexible member. When the fistulization device is inserted into a patient through an incision such that the at least one first elastomeric arm extends past a distal opening of the incision, the at least one first elastomeric arm is configured to return to its relaxed state and prevent proximal displacement of the device within the incision, and the at least one second elastomeric arm is configured to return to its relaxed state and prevent distal displacement of the device within the incision. When the at least one first elastomeric arm and the at least one second elastomeric arm are deformed to facilitate insertion into a patient, mechanical energy is stored in the polymer chains of their material due to reversible elastic stretching. Upon removal of the external, deforming force (i.e., from the delivery tool lumen) on the at least one first elastomeric arm, the stored strain energy drives the material of the at least one first elastomeric arm back toward its original shape. Similarly, upon removal of the external, deforming force on the at least one second elastomeric arm, the stored strain energy drives the material of the at least one second elastomeric arm back toward its original shape.

The flexible member is configured to allow fluid migration past the flexible member and through a proximal opening of the incision, for example via the at least one longitudinally extending groove.

According to some embodiments of the present invention a kit for treating cysts and abscesses includes a fistulization device, and a delivery tool for positioning the fistulization device within a patient. The fistulization device includes a flexible member having opposite proximal and distal ends, at least one first elastomeric arm at the flexible member distal end, and at least one second elastomeric arm at the flexible member proximal end. The at least one first elastomeric arm has a curved configuration when in a relaxed state, and the at least one second elastomeric arm has a curved configuration when in a relaxed state.

The delivery tool includes a barrel having a distal end, a proximal end, and a lumen extending from the barrel distal end to the barrel proximal end. The fistulization device is deformable so as to be insertable into the lumen such that the at least one first elastomeric arm extends longitudinally away from the distal end of the flexible member in a non-relaxed state, and such that the at least one second elastomeric arm extends longitudinally away from the proximal end of the flexible member in a non-relaxed state. A plunger is slidably mounted within the barrel lumen and is configured to deploy the fistulization device through the barrel distal end and into an incision in the patient. In some embodiments, the barrel distal end includes a scalpel for forming the incision in the patient or is otherwise configured to form the incision in the patient.

According to other embodiments of the present invention, a method of treating a cyst or abscess includes making an incision in a patient at a location of the cyst or abscess. A distal end of a delivery tool is positioned at a proximal opening of the incision. The delivery tool includes a barrel having a distal end, a proximal end, a lumen extending from the barrel distal end to the barrel proximal end, and a plunger slidably mounted within the barrel lumen. A fistulization device is located within the barrel lumen and includes a flexible member having opposite proximal and distal ends, at least one first elastomeric arm at the flexible member distal end, and at least one second elastomeric arm at the flexible member proximal end. The at least one first elastomeric arm has a curved configuration when in a relaxed state, and the at least one second elastomeric arm has a curved configuration when in a relaxed state. The fistulization device is located within the barrel lumen such that the at least one first elastomeric arm extends longitudinally away from the distal end of the flexible member, and such that the at least one second elastomeric arm extends longitudinally away from the proximal end of the flexible member.

The plunger is moved toward the barrel distal end to deploy the fistulization device into the incision such that the at least one first elastomeric arm is positioned past a distal opening of the incision and returns to its relaxed state (because of stored strain energy in the material) so as to prevent inadvertent proximal displacement of the device within the incision, and such that the at least one second arm is positioned adjacent the proximal opening of the incision and returns to its relaxed state (because of stored strain energy in the material) so as to prevent inadvertent distal displacement of the device within the incision. Once installed, the fistulization device allows a fistula tract to form so that fluid can drain from the cyst or abscess along the flexible member thereof and out the proximal opening of the incision.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate various embodiments of the present invention. The drawings and description together serve to fully explain embodiments of the present invention.

FIG. 1 is a plan view of a fistulization device for treating cysts and abscesses according to some embodiments of the present invention.

FIG. 2 is an illustration of a kit for treating cysts and abscesses according to some embodiments of the present invention. The kit includes a delivery tool and the fistulization device of FIG. 1 within the barrel of the delivery tool with the first and second arms of the fistulization device in an elongated, non-relaxed state.

FIG. 3 is an enlarged view of the distal end of the barrel of the delivery tool of FIG. 2 and illustrating the fistulization device with the first and second arms in an elongated, non-relaxed state and partially extending from the distal end of the barrel.

FIG. 4 is an illustration of the fistulization device of FIG. 1 installed within an incision at the location of a cyst or abscess in a patient.

FIGS. 5A-5E illustrate a method of treating a cyst or abscess with the fistulization device of FIG. 1.

FIG. 6 is an illustration of a kit for treating cysts and abscesses according to other embodiments of the present invention. The kit includes a delivery tool and a fistulization device within the barrel of the delivery tool.

FIGS. 7A-7E illustrate a method of treating a cyst or abscess with the fistulization device of FIG. 6.

### DETAILED DESCRIPTION OF THE INVENTION

The present invention will now be described more fully hereinafter with reference to the accompanying figures, in which embodiments of the present invention are shown. The present invention may, however, be embodied in many different forms and should not be construed as limited to the embodiments set forth herein.

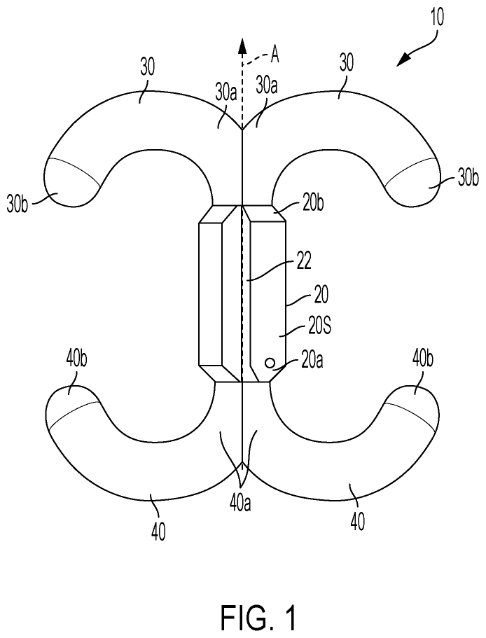

Referring to FIG. 1, a fistulization device 10 for treating a cyst or abscess, according to some embodiments of the present invention, is illustrated. The illustrated fistulization device 10 is a flexible device formed from soft, elastomeric material, such as silicone, and includes a central member 20 having opposite proximal and distal ends 20a, 20b. The central member 20 has a generally cylindrical configuration. However, other configurations are possible. For example, the central member 20 may have a polygonal cross-sectional configuration.

The central member 20 may include at least one longitudinally extending (i.e., extending along a longitudinal axis A of the central member 20) groove 22 in an outer surface 20S of the central member 20. For example, in the illustrated embodiment, the central member 20 includes a longitudinally extending groove 22 on the illustrated side of the central member 20. Another longitudinally extending groove may be in the central member on the opposite side. Moreover, the central member 20 may have a plurality of longitudinally extending grooves circumferentially spaced apart around the central member 20. Each longitudinally extending groove, such as groove 22, is configured to facilitate the flow of fluid from a cyst or abscess outward from a patient through the incision in which the fistulization device 10 is positioned.

A pair of first elastomeric arms 30 extend outward from the central member distal end 20b, and a pair of second elastomeric arms 40 extend outward from the central member proximal end 20a, as illustrated. Each of the first arms 30 has opposite first and second ends 30a, 30b. The first ends 30a of the first arms 30 are connected together and to the distal end 20b of the central member 20, as illustrated. Similarly, each of the second arms 40 has opposite first and second ends 40a, 40b. The first ends 40a of the second arms 40 are connected together and to the proximal end 20a of the central member 20, as illustrated. The second or free end 30b, 40b of each of the first and second arms 30, 40 is rounded or blunt so as not to cause tissue damage when inserted into a patient. The curved configuration of the first and second arms 30, 40 is an advantageous feature of the present invention because the fistulization device 10 can stay in place within a patient without “tissue piercing/grabbing” features like barbs, etc. Moreover, the outer surface of the fistulization device, including the central member 20, is typically smooth with no sharp edges. Overall, the fistulation device 10 has an atraumatic profile so that it can pass through an incision in a patient with minimal risk of snagging, lacerating, or otherwise damaging patient tissue.

FIG. 1 illustrates the first and second arms 30, 40 in a relaxed state, and wherein each has a curved configuration in the relaxed state. In the illustrated embodiment, each first and second arm 30, 40 has a generally semi-circular shape when in the relaxed state. However, other shapes are possible. Moreover, although a pair of first and second arms 30, 40 are illustrated, in other embodiments, different numbers of first and second arms 30, 40, including even a single first and a single second arm, may be utilized.

In the illustrated embodiment, the pair of first arms 30 are arranged in adjacent opposing relationship, and the pair of second arms 40 are arranged in adjacent opposing relationship. As such, the pair of first arms 30 curve away from each other when in their respective relaxed states, and the pair of second arms 40 curve away from each other when in their respective relaxed states. In the illustrated embodiment, the first arms 30, second arms 40, and central member 20 are substantially within the same plane when the device 10 is in a relaxed state. However, other configurations are possible and it is not a requirement of the present invention that the first arms 30, second arms 40, and central member 20 are substantially within the same plane when the device 10 is in a relaxed state.

In the illustrated embodiment, the second or free end 30b of each first arm 30 and the second or free end 40b of each second arm 40 on the same side of the central member 20 are curved toward each other when in their respective relaxed states. However, in other embodiments, the second or free ends 30b, 40b may curve in a different direction.

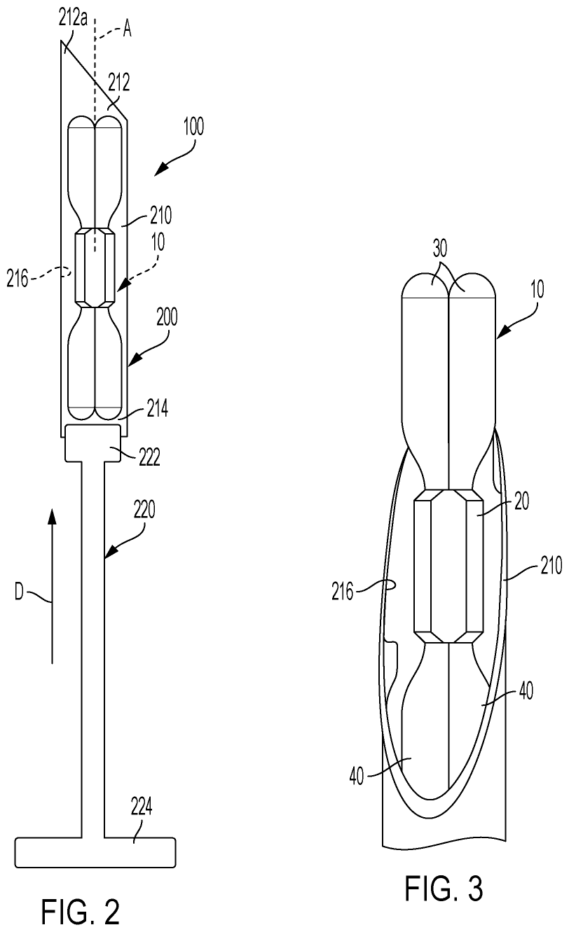

The fistulization device 10 of the present invention is flexible (i.e., deformable) so as to be insertable into a lumen of a delivery tool for insertion into a patient. For example, FIG. 2 illustrates a kit 100 for treating cysts and abscesses according to some embodiments of the present invention. The kit 100 includes a delivery tool 200 and the fistulization device 10 of FIG. 1 within a barrel 210 of the delivery tool 200 with the first and second arms 30, 40 of the fistulization device 10 deformed into an elongated, non-relaxed state. This configuration allows the fistulization device 10 to be inserted within a small, narrow incision in a patient, as will be described below. Mechanical energy (i.e., strain energy) is stored in the polymer chains of the material of the first and second elastomeric arms 30, 40 when in the elongated, non-relaxed state. This strain energy drives the first and second arms 30, 40 back toward their curved, relaxed state upon removal from the delivery tool 200.

The barrel 210 of the illustrated delivery tool includes a distal end 212, a proximal end 214, and a lumen 216 extending from the barrel distal end 212 to the barrel proximal end 214. The fistulization device 10 is insertable into the lumen 216 of the barrel 210 such that the pair of first arms 30 extend longitudinally (i.e., along longitudinal axis A) away from the distal end 20b of the central member 20 (i.e., the first arms 30 are in a non-relaxed state), and such that the pair of second arms 40 extend longitudinally (i.e., along longitudinal axis A) away from the proximal end 20a of the central member 20 (i.e., the second arms 40 are in a non-relaxed state).

A plunger 220 is slidably mounted within the barrel lumen 216 and has an elongate configuration with a distal end portion 222 and a proximal end portion 224. The distal end portion 222 of the plunger 220 is configured to engage the fistulization device 10 within the barrel lumen 216 and push the fistulization device 10 out of the barrel distal end 212 upon movement of the plunger 220 in direction D, for example, by a healthcare provider. The plunger proximal end portion 224 is configured as a thumb press that can be pressed by a user to move the plunger distal end portion 222 toward the barrel distal end 212, and can be gripped and pulled by a user to move the plunger distal end portion 222 toward the barrel proximal end 214. The illustrated barrel 200 and plunger 220 may have various other shapes and configurations. Embodiments of the present invention are not limited to the illustrated shape and configuration of the barrel 200 and plunger 220.

In some embodiments, the barrel distal end 212 may include a scalpel or may be configured as a scalpel for forming an incision in the patient. For example, a portion 212a of the barrel distal end 212 may be sufficiently sharp to serve the function of a blade of a scalpel.

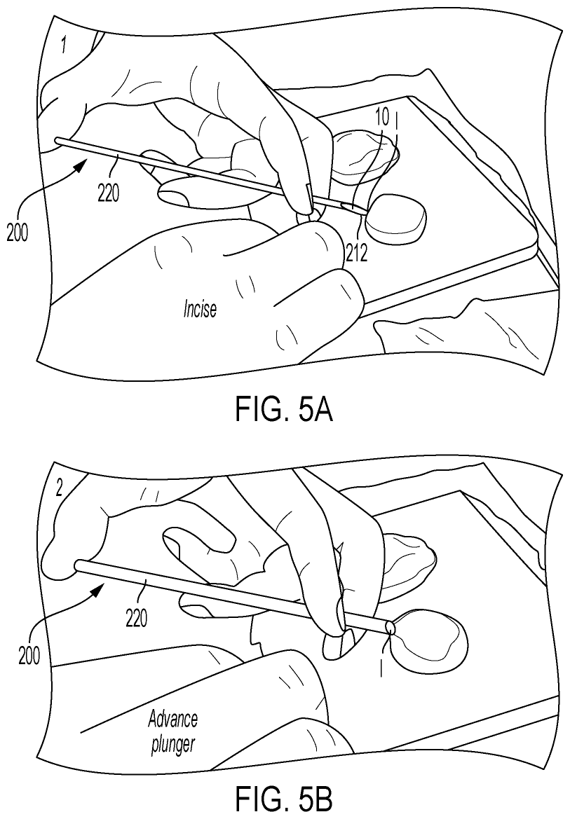

FIG. 4 is an illustration of the fistulization device 10 of FIG. 1 installed within an incision I (e.g., via the delivery tool 200 of FIG. 2) at the location of a cyst or abscess in a patient. The fistulization device 10 is inserted through the incision I such that the pair of first arms 30 (in an elongated, non-relaxed configuration) extend past the distal opening Id of the incision I and then return to their relaxed state. In their relaxed state, the first arms 30 are curved such that the second or free ends 30b thereof can engage the skin of the patient and, thereby, prevent inadvertent proximal displacement of the fistulization device 10 within the incision I. The pair of second arms, upon leaving a delivery device (e.g., delivery tool 200 of FIG. 2) return to their relaxed state. In their relaxed state, the second arms 40 are curved such that the second or free ends 40b thereof can engage the skin of the patient and, thereby, prevent inadvertent distal displacement of the device 10 within the incision I. The fistulization device 10 passively keeps the incision I open, allowing a fistula to form. The one or more longitudinally extending grooves 22 in the central member 20 are configured to allow fluid migration past the central member 20 and through the proximal opening Ip of the incision I.

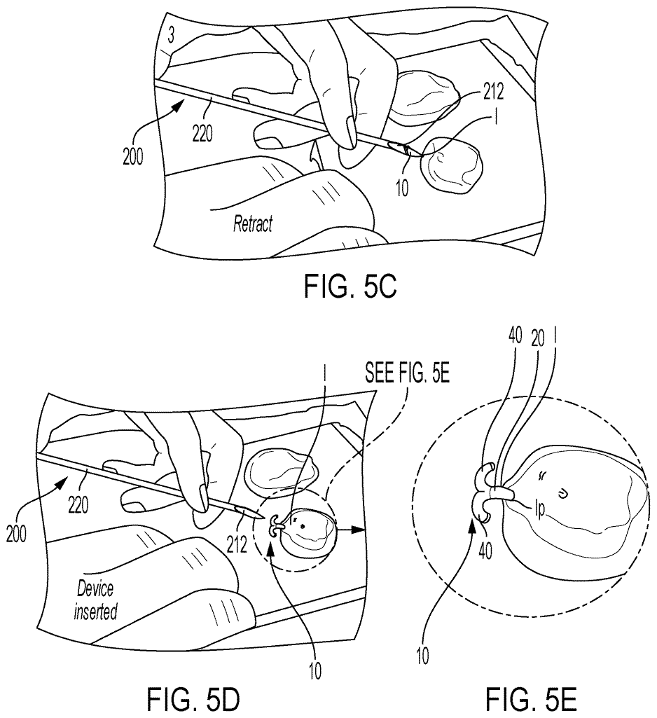

FIGS. 5A-5E illustrate a method of treating a cyst or abscess with the fistulization device 10 of FIG. 1. In FIG. 5A, a delivery device (e.g., the delivery tool 200 of FIG. 2) is utilized to make an incision I in a patient at the location of a cyst or abscess. In FIG. 5B, the plunger 220 of the delivery device 200 is operated to push the fistulization device 10 out of the delivery device and into the incision I. In FIG. 5C, the delivery device is retracted from the incision I after the fistulization device 10 has been placed within the incision I. FIGS. 5D and 5E illustrate the inserted fistulization device 10. The first arms 30 (not shown) are in their relaxed state and can engage the skin of the patient at the distal opening of the incision I to prevent inadvertent proximal displacement of the fistulization device 10 within the incision I, and the second arms 40 are in their relaxed state and can engage the skin of the patient at the proximal opening Ip of the incision I and, thereby, prevent inadvertent distal displacement of the device 10 within the incision I.

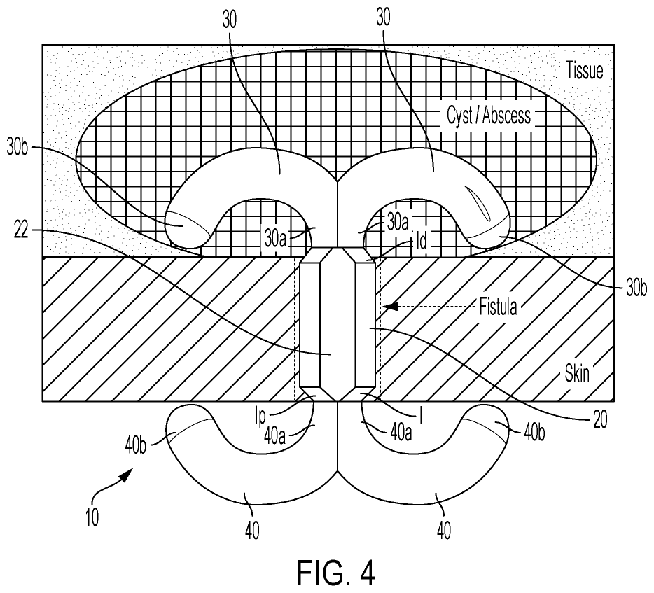

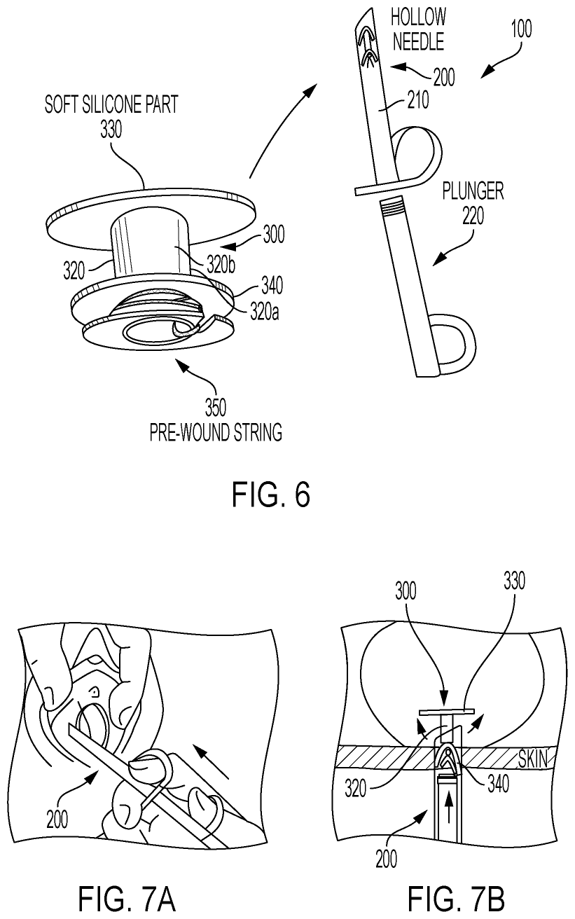

FIG. 6 is an illustration of a kit 100 for treating cysts and abscesses according to other embodiments of the present invention. The kit 100 includes a delivery tool 200 and a different fistulization device 300 within the barrel 210 of the delivery tool 200. The illustrated fistulization device 300 is a flexible, low-profile device (e.g., formed from silicone or other similar soft, flexible material) in the shape of a bobbin that includes a central member 320, a first elastomeric flange 330 at the central member distal end 320b, and second elastomeric flange 340 at the central member proximal end 320a. Similar to the fistulization device 10 of FIGS. 1-4, the illustrated fistulization device 300 is configured to keep an incision open, thereby allowing a fistula to form.

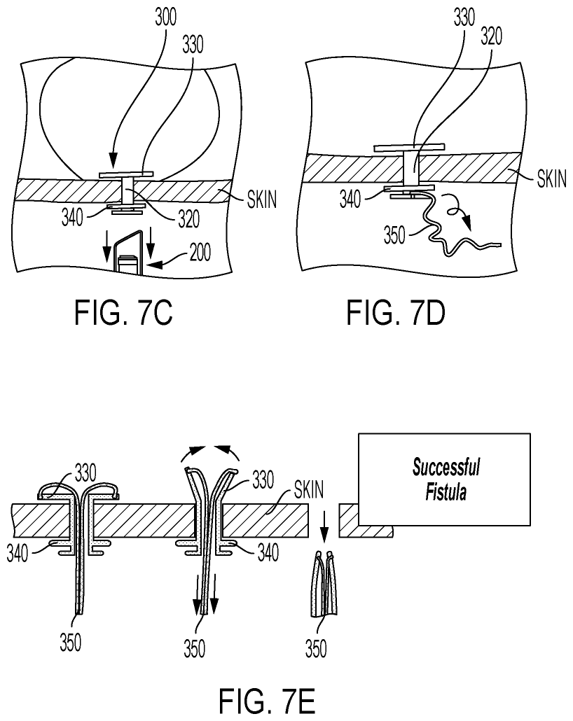

One or more pre-wound strings 350 are attached to the fistulization device 300 adjacent to the second flange 340, as illustrated. The one or more pre-wound strings 350 facilitate removal, of the fistulization device 300 from a patient, similar in technique to intrauterine device (IUD) removal, as will be described below with respect to FIGS. 7A-7E.

In FIG. 7A, a healthcare provider uses a needle tip of the delivery device 200 to lance into a gland through the skin, then pushes the plunger to begin insertion of the fistulization device 300. In FIG. 7B the plunger partially ejects the fistulization device 300, allowing the device's internal flange 330 to expand inside the gland. In FIG. 7C, the delivery tool is removed, allowing the fistulization device's external flange 340 to fold out flat.

Removal of the device 300 is illustrated in FIGS. 7D-7E. The strings 350 are unwound causing the internal flange to fold up so the device can be removed from the fistula tract.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity. The term “and/or” includes any and all combinations of one or more of the associated listed items.

Like numbers refer to like elements throughout. In the figures, certain components or features may be exaggerated for clarity, and broken lines may illustrate optional features or operations unless specified otherwise. In addition, the sequence of operations (or steps) is not limited to the order presented in the figures and/or claims unless specifically indicated otherwise. Features described with respect to one figure or embodiment can be associated with another embodiment or figure although not specifically described or shown as such.

It will be understood that when an element is referred to as being “on” another element, it can be directly on the other element or intervening elements may also be present. In contrast, when an element is referred to as being “directly on” another element, there are no intervening elements present. It will also be understood that when an element is referred to as being “connected”, “coupled”, “responsive”, or variants thereof to another element, it can be directly connected, coupled or responsive to the other element or intervening elements may be present. In contrast, when an element is referred to as being “directly connected”, “directly coupled”, “directly responsive”, or variants thereof to another element, there are no intervening elements present. Other words used to describe the relationship between elements should be interpreted in a like fashion (i.e., “between” versus “directly between”, “adjacent” versus “directly adjacent”, etc.).

As used herein, the singular forms “a”, “an” and “the” are intended to include the plural forms as well, unless the context clearly indicates otherwise. As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.,” which derives from the Latin phrase “exempli gratia,” may be used to introduce or specify a general example or examples of a previously mentioned item, and is not intended to be limiting of such item. The common abbreviation “i.e.,” which derives from the Latin phrase “id est,” may be used to specify a particular item from a more general recitation.

It will be understood that although the terms first, second, third, etc., may be used herein to describe various elements/operations, these elements/operations should not be limited by these terms. These terms are only used to distinguish one element/operation from another element/operation. Thus, a first element/operation in some embodiments could be termed a second element/operation in other embodiments without departing from the teachings of the present invention. The same reference numerals or the same reference designators denote the same or similar elements throughout the specification.

Relative terms such as “below” or “above” or “upper” or “lower” or “horizontal” or “vertical” may be used herein to describe a relationship of one element, layer or region to another element, layer or region as illustrated in the figures. It will be understood that these terms are intended to encompass different orientations of the device in addition to the orientation depicted in the figures.

The terms “about” and “approximately”, as used herein with respect to a value or number, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

What is claimed is:

1. A fistulization device for treating cysts and abscesses, the device comprising:
a flexible member having opposite proximal and distal ends;
at least one first elastomeric arm at the flexible member distal end, wherein the at least one first elastomeric arm has a curved configuration when in a relaxed state; and
at least one second elastomeric arm at the flexible member proximal end, wherein the at least one second elastomeric arm has a curved configuration when in a relaxed state.

2. The device of claim 1, wherein the at least one first elastomeric arm comprises a pair of first elastomeric arms, wherein the at least one second elastomeric arm comprises a pair of second elastomeric arms, wherein the pair of first elastomeric arms are arranged in adjacent opposing relationship, and wherein the pair of second elastomeric arms are arranged in adjacent opposing relationship.

3. The device of claim 1, wherein the at least one first elastomeric arm, the at least one second elastomeric arm, and the flexible member are substantially within a same plane.

4. The device of claim 2, wherein the pair of first elastomeric arms curve away from each other when in the relaxed state, and wherein the pair of second elastomeric arms curve away from each other when in the relaxed state.

5. The device of claim 1, wherein the flexible member comprises at least one longitudinally extending groove in an outer surface of the flexible member.

6. The device of claim 5, wherein the at least one longitudinally extending groove comprises a plurality of longitudinally extending grooves circumferentially spaced apart around the flexible member.

7. The device of claim 1, wherein a free end of the at least one first elastomeric arm is blunt, and wherein a free end of the at least one second elastomeric arm is blunt.

8. The device of claim 1, wherein a free end of the at least one first elastomeric arm and a free end of the at least one second elastomeric arm are curved toward each other when in their respective relaxed states.

9. The device of claim 1, wherein the device is insertable into a lumen of a delivery tool such that the at least one first elastomeric arm extends longitudinally away from the distal end of the flexible member, and such that the at least one second elastomeric arm extends longitudinally away from the proximal end of the flexible member.

10. The device of claim 1, wherein, when the device is inserted into a patient through an incision such that the at least one first elastomeric arm extends past a distal opening of the incision, the at least one first elastomeric arm is configured to return to its relaxed state because of stored strain energy and prevent inadvertent proximal displacement of the device within the incision, the at least one second elastomeric arm is configured to return to its relaxed state because of stored strain energy and prevent inadvertent distal displacement of the device within the incision, and the flexible member is configured to allow fluid migration past the flexible member and through a proximal opening of the incision.

11. A kit for treating cysts and abscesses, the kit comprising:
a fistulization device, comprising:
a flexible member having opposite proximal and distal ends;
at least one first elastomeric arm at the flexible member distal end, wherein the at least one first elastomeric arm has a curved configuration when in a relaxed state; and
at least one second elastomeric arm at the flexible member proximal end, wherein the at least one second elastomeric arm has a curved configuration when in a relaxed state; and
a delivery tool for positioning the fistulization device within a patient, the delivery tool comprising:
a barrel having a distal end, a proximal end, and a lumen extending from the barrel distal end to the barrel proximal end, wherein the fistulization device is insertable into the lumen such that the at least one first elastomeric arm extends longitudinally away from the distal end of the flexible member in a non-relaxed state, and such that the at least one second elastomeric arm extends longitudinally away from the proximal end of the flexible member in a non-relaxed state; and
a plunger slidably mounted within the barrel lumen, wherein the plunger is configured to deploy the fistulization device through the barrel distal end and into an incision in the patient.

12. The kit of claim 11, wherein the barrel distal end is configured to form the incision in the patient.

13. The kit of claim 11, wherein the at least one first elastomeric arm comprises a pair of first elastomeric arms, wherein the at least one second elastomeric arm comprises a pair of second elastomeric arms, wherein the pair of first elastomeric arms are arranged in adjacent opposing relationship, and wherein the pair of second elastomeric arms are arranged in adjacent opposing relationship.

14. The kit of claim 11, wherein the at least one first elastomeric arm, the at least one second elastomeric arm, and the flexible member are substantially within a same plane.

15. The kit of claim 13, wherein the pair of first elastomeric arms curve away from each other when in their relaxed state, and wherein the pair of second elastomeric arms curve away from each other when in their relaxed state.

16. The kit of claim 11, wherein the flexible member comprises at least one longitudinally extending groove in an outer surface thereof.

17. The kit of claim 16, wherein the at least one longitudinally extending groove comprises a plurality of longitudinally extending grooves circumferentially spaced apart around the flexible member.

18. The kit of claim 11, wherein a free end of the at least one first elastomeric arm is blunt, and wherein a free end of the at least one second elastomeric arm is blunt.

19. The kit of claim 11, wherein a free end of the at least one first elastomeric arm and a free end of the at least one second elastomeric arm are curved toward each other when in their respective relaxed states.

20. A method of treating cysts and abscesses, the method comprising:
making an incision in a patient at a location of a cyst or abscess;
locating a distal end of a delivery tool at a proximal opening of the incision, wherein the delivery tool comprises a barrel having a distal end, a proximal end, a lumen extending from the barrel distal end to the barrel proximal end, and a plunger slidably mounted within the barrel lumen, wherein a fistulization device is located within the barrel lumen and comprises:
a flexible member having opposite proximal and distal ends;
at least one first elastomeric arm at the flexible member distal end, wherein the at least one first elastomeric arm has a curved configuration when in a relaxed state; and
at least one second elastomeric arm at the flexible member proximal end, wherein the at least one second elastomeric arm has a curved configuration when in a relaxed state;
wherein the fistulization device is located within the barrel lumen such that the at least one first elastomeric arm extends longitudinally away from the distal end of the flexible member, and such that the at least one second elastomeric arm extends longitudinally away from the proximal end of the flexible member; and
moving the plunger toward the barrel distal end to deploy the fistulization device into the incision such that the at least one first elastomeric arm is positioned past a distal opening of the incision and returns to its relaxed state so as to prevent inadvertent proximal displacement of the device within the incision, and such that the at least one second arm is positioned adjacent the proximal opening of the incision and returns to its relaxed state so as to prevent inadvertent distal displacement of the device within the incision.
